(19) United States
(12) Patent Application Publication
(10) Pub. No.: US 2026/0224193 A1
(43) Pub. Date: Aug. 6, 2026

(54) INTRACARDIAC ECHOCARDIOGRAPHY CATHETER WITH MODE ACTIVATION CONTROLS

(71) Applicant: Boston Scientific Scimed, Inc., Maple Grove, MN (US)

(72) Inventors: Binh C. Tran, St. Paul, MN (US); Christian Balkovec, Cambridge (CA)

(21) Appl. No.: 19/465,084

(22) Filed: Jan. 30, 2026

Related U.S. Application Data
(60) Provisional application No. 63/752,524, filed on Jan. 31, 2025.

(51) Int. Cl. A61B 8/12; A61B 8/00; A61B 8/08; G06T 7/00

(52) U.S. Cl. CPC A61B 8/12; A61B 8/0883; A61B 8/445; A61B 8/4488; A61B 8/468; A61B 8/488; A61B 8/5215; A61B 8/54; G06T 7/0012; A61B 2560/0462; A61B 2562/0204; A61B 2562/028; A61B 2562/046; G06T 2207/10132; G06T 2207/30101

(57) ABSTRACT

A medical imaging catheter to be extended into a patient’s heart is disclosed. The medical imaging catheter includes a shaft having a proximal region and an opposite distal region, an ultrasound imaging module coupled to the distal region to be extended into the patient’s heart, and a handle assembly coupled to the proximal region. The handle assembly includes a mode controller to switch the medical imaging catheter into one of several modes and to adjust an aspect of the mode.

### CROSS REFERENCE TO RELATED APPLICATIONS

This application claims priority to U.S. Provisional Application No. 63/752,524 entitled “INTRACARDIAC ECHOCARDIOGRAPHY CATHETER WITH MODE ACTIVATION CONTROLS,” filed January 31, 2025, which is hereby incorporated by reference in its entirety.

### TECHNICAL FIELD

The present disclosure relates to medical systems and methods for facilitating visualization of tissue from within a patient. More specifically, the present disclosure relates to medical systems, catheters, and methods having ultrasonic transducer devices.

### BACKGROUND

Intracardiac echocardiography (ICE) is an ultrasonic imaging modality that has become a common aspect of several percutaneous interventional and electrophysiology procedures. ICE can provide high resolution and real-time visualizations of cardiac structures, continuous monitoring of catheter location within the heart, and early recognition of procedural complications such as pericardial effusion or thrombus formation. An ICE catheter typically is inserted in a vein, such as via standard femoral venous introducer into the femoral vein, and then passed from the vein into the heart alongside other tools. Another common ultrasonic imaging modality includes transesophageal echocardiography (TEE) and fluoroscopy to image and visualize tools within the heart, which requires an anesthesiologist and additional echocardiologist to keep the patient under general anesthesia and to manipulate an ultrasonic probe within the airway. In contrast to TEE, ICE can be performed by the primary operator of the interventional procedure under conscious sedation, without endotracheal intubation, and with a reduced risk of esophageal trauma. Additionally, ICE reduces fluoroscopy exposure for both the patient and the operator. For these reasons, ICE is a preferred imaging modality in certain procedures such as atrial septal defect closure and catheter ablation of cardiac arrhythmias and includes an emerging role in other procedures such as mitral valvuloplasty, transcatheter aortic valve replacement, and left atrial appendage closure.

Two forms of ICE are available. Radial or rotational ICE uses a single piezoelectric crystal mounted on a tip of a six to ten French catheter. A rotating transducer provides cross-sectional images in a radial plane perpendicular to a longitudinal axis of the catheter. Rotational ICE operates at imaging frequencies that are useful for near-field imaging of up to six or eight centimeters but is limited for far-field imaging. Phased-array ICE uses a multi-element transducer, such as a sixty-four-element transducer, mounted on a distal end of an eight to ten French steerable catheter that can often be deflected in four directions including anterior, posterior, right, and left. The device provides a wedge-shaped image, such as a ninety-degree sector plane, from the side of the catheter that can be displayed on a conventional ultrasound workstation. When contrasted with mechanical rotational ICE systems, phased-array ICE can provide for greater image-depth penetration (up to 15 cm), greater maneuverability, and the ability to acquire Doppler and color flow imaging.

### SUMMARY

In Example 1, a medical imaging catheter configured to be extended into a patient’s heart, the medical imaging catheter comprising: a shaft having a proximal region and an opposite distal region; an ultrasound imaging module coupled to the distal region and configured to be extended into the patient’s heart; and a handle assembly coupled to the proximal region, the handle assembly including a mode controller configured to switch the medical imaging catheter into one of a plurality of modes and to adjust an aspect of the mode.

In Example 2, the medical imaging catheter of Example 1, wherein the medical imaging catheter is an intracardiac echocardiography (ICE) device.

In Example 3, the medical imaging catheter of any of Examples 1 and 2, wherein the mode controller includes a mode actuator and an adjuster dial.

In Example 4, the medical imaging catheter of Example 3, wherein the mode actuator includes a pushbutton and the adjuster dial includes a ring rotatable with respect to the handle assembly.

In Example 5, the medical imaging catheter of any of Examples 1-4, wherein the medical imaging catheter is configured to be coupled to a medical imaging console as a medical imaging system.

In Example 6, the medical imaging catheter of Example 5, wherein the medical imaging console includes a controller configured to apply a plurality of features to an ultrasound image.

In Example 7, the medical imaging catheter of Example 6, wherein the controller is configured to respond to a signal from the mode controller.

In Example 8, the medical imaging catheter of any of Examples 6 and 7, wherein the plurality of features includes a plurality of ultrasound modes.

In Example 9, the medical imaging catheter of any of Examples 6-8, wherein the plurality of features includes an anatomical feature detector mode configured to automatically determine an anatomical feature from an ultrasound image via an artificial intelligence.

In Example 10, the medical imaging catheter of Example 9, wherein the anatomical feature detector mode includes a pulmonary vein detector mode configure to apply labels to a visualization of the pulmonary veins from an ultrasound image.

In Example 11, the medical imaging catheter of any of Examples 7-10, wherein the mode controller switches between a subset of the plurality of features.

In Example 12, the medical imaging catheter of Examples 11, wherein the mode controller toggles between an initial feature and a first feature.

In Example 13, the medical imaging catheter of Examples 12, wherein the initial feature is a brightness mode and the first feature is a Doppler mode.

In Example 14, the medical imaging catheter of Example 13, wherein the mode controller is configured to adjust a center frequency while the controller is configured in the brightness mode and configured to adjust a Doppler window while the controller is configured in the Doppler mode.

In Example 15, the medical imaging catheter of any of Examples 11-14, wherein the subset of features is less than the plurality of features.

In Example 16, a medical imaging catheter configured to be extended into a patient’s heart, the medical imaging catheter comprising: a shaft having a proximal region and an opposite distal region; an ultrasound imaging module coupled to the distal region and configured to be extended into the patient’s heart; and a handle assembly coupled to the proximal region, the handle assembly including a mode controller configured to switch the medical imaging catheter into one of a plurality of modes and to adjust an aspect of the mode.

In Example 17, the medical imaging catheter of Example 16, wherein the medical imaging catheter is an intracardiac echocardiography (ICE) device.

In Example 18, the medical imaging catheter of Example 16, wherein the mode controller includes a mode actuator and an adjuster dial.

In Example 19, the medical imaging catheter of Example 18, wherein the mode actuator includes a pushbutton and the adjuster dial includes a ring rotatable with respect to the handle assembly.

In Example 20, the medical imaging catheter of Example 16, wherein the medical imaging catheter is configured to be coupled to a medical imaging console as a medical imaging system.

In Example 21, the medical imaging catheter of Example 20, wherein the medical imaging console includes controller configured to apply a plurality of features to an ultrasound image.

In Example 22, the medical imaging catheter of Example 21, wherein the controller is configured to respond to a signal from the mode controller.

In Example 23, the medical imaging catheter of Example 22, wherein the mode controller switches between a subset of the plurality of features.

In Example 24, the medical imaging catheter of Example 23, wherein the mode controller toggles between an initial feature and a first feature.

In Example 25, the medical imaging catheter of Example 24, wherein the initial feature is a brightness mode and the first feature is a Doppler mode.

In Example 26, the medical imaging catheter of Example 25, wherein the mode controller is configured to adjust a center frequency while the controller is configured in the brightness mode and configured to adjust a Doppler window while the controller is configured in the Doppler mode.

In Example 27, the medical imaging catheter of Example 24, wherein the subset of features is less than the plurality of features.

In Example 28, the medical imaging catheter of Example 21, wherein the plurality of features includes a plurality of ultrasound modes.

In Example 29, the medical imaging catheter of Example 21, wherein the plurality of features includes an anatomical feature detector mode configured to automatically determine an anatomical feature from an ultrasound image via an artificial intelligence.

In Example 30, the medical imaging catheter of Example 29, wherein the anatomical feature detector mode includes a pulmonary vein detector mode configure to apply labels to a visualization of the pulmonary veins from an ultrasound image.

In Example 31, an intracardiac echocardiography (ICE) catheter configured to be extended into a patient’s heart, the ICE catheter comprising: a shaft having a proximal region and an opposite distal region; an ultrasound imaging module coupled to the distal region and configured to be extended into the patient’s heart, the ultrasound imaging module including a piezoelectric micro-machined ultrasound transducer (pMUT) connected to a flex circuit configured for transmission of electrical signals along the shaft; and a handle assembly coupled to the proximal region, the handle assembly including a mode controller configured to switch the ICE catheter into one of a plurality of modes and to adjust an aspect of the mode, the mode controls including an electronic element within the handle assembly and an actuation mechanism disposed on the handle assembly.

In Example 32, the ICE catheter of Example 31, wherein the mode controller includes a mode actuator and an adjuster dial.

In Example 33, the ICE catheter of Example 32, wherein the mode actuator includes a pushbutton and the adjuster dial includes a ring rotatable with respect to the handle assembly.

In Example 34, a method for use with a medical imaging system having a medical imaging catheter having an ultrasound imaging module configured to be extended into a patient’s heart and a handle assembly coupled to the imaging module, the handle assembly including a mode controller having a mode actuator and an adjuster dial, the method comprising: applying a plurality of features to an ultrasound image, the plurality of features including a brightness mode and a Doppler mode; and switching between a subset of the plurality of features in response to a signal from the mode actuator; and adjusting a center frequency while in the brightness mode and adjusting a Doppler window while in the Doppler mode in response to a signal from the adjuster dial.

In Example 35, the method of Example 34, wherein the plurality of features includes an anatomical feature detector mode configured to automatically determine an anatomical feature from an ultrasound image via an artificial intelligence.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram a medical imaging system used while treating a patient such as treating a heart of the patient.

FIG. 2 is a schematic diagram of a region of a medical imaging catheter of the medical imaging system of FIG. 1.

FIG. 3 is a schematic diagram of an ultrasound transducer assembly for use in a medical imaging catheter of the medical imaging system of FIG. 1.

FIG. 4 is a schematic diagram of a controller of a medical imaging console of the medical imaging system of FIG. 1.

FIG. 5 is a schematic diagram of another region of a medical imaging catheter of the medical imaging system of FIG. 1.

FIG. 6 is a diagram of a partially sectioned heart with the region of the medical imaging catheter in a right atrium of the heart.

FIG. 7A is a diagram of a first visualization produced from an ultrasound image of the medical imaging system of FIG. 1.

FIG. 7B is a diagram of a second visualization produced from an ultrasound image of the medical imaging system of FIG. 1.

FIG. 8 is a diagram of a third visualization produced from an ultrasound image of the medical imaging system of FIG. 1.

FIG. 9 is a block diagram of a method of the controller of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Sound waves in medical ultrasound imaging are generated by piezoelectric crystal elements in ultrasound transducers. The elements vibrate and generate sound waves with specific frequencies when exposed to a voltage. As the sound waves propagate through tissue, they encounter various tissue interfaces. At these interfaces, sound can either be reflected, scattered, refracted, or absorbed. The loss of energy associated with this is referred to as attenuation. Higher frequencies experience greater attenuation in tissues and therefore cannot penetrate as deeply as lower frequencies.

As the sound wave travels through tissue, some echoes are reflected back towards the transducer. As the sound waves return, the soundwaves interact with the elements in the ultrasound transducer, causing vibration and deformation. This, in turn, causes voltage in the transducer, which is relayed back to a controller. The controller is then able to process this information into an image. Because the average propagation velocity of sound waves in soft tissue is 1540 m/s, the controller uses the time it takes sound waves to return to the transducer to determine the depth or distance of objects. Similarly, the amplitude of the waves returning to the transducer from a particular object informs the brightness of that object as presented on a visualization by a display.

In one example, greyscale or brightness mode (B-Mode) imaging utilizes the amplitude of reflected echoes to plot information into a two-dimensional image. Doppler ultrasonography analyzes the frequency of the returning echo to determine relative motion based on the Doppler effect. The Doppler effect states that when a sonic source is moving towards or away from a stationary listening device, the relative frequency heard by the device will be shifted according to the velocity of the source. In cases where the source is moving away from the listening device, the frequency will be shifted lower, and in cases where the source is moving towards the listening device, the frequency is shifted higher. In the case of ultrasound, the transducer transmits sound waves at a given frequency to a moving object, such as a red blood cell in a flow of blood. This object then reflects a portion of the sound wave back as an echo. As the sound wave returns to the transducer, the frequency will be altered based on the speed and direction of the traveling object.

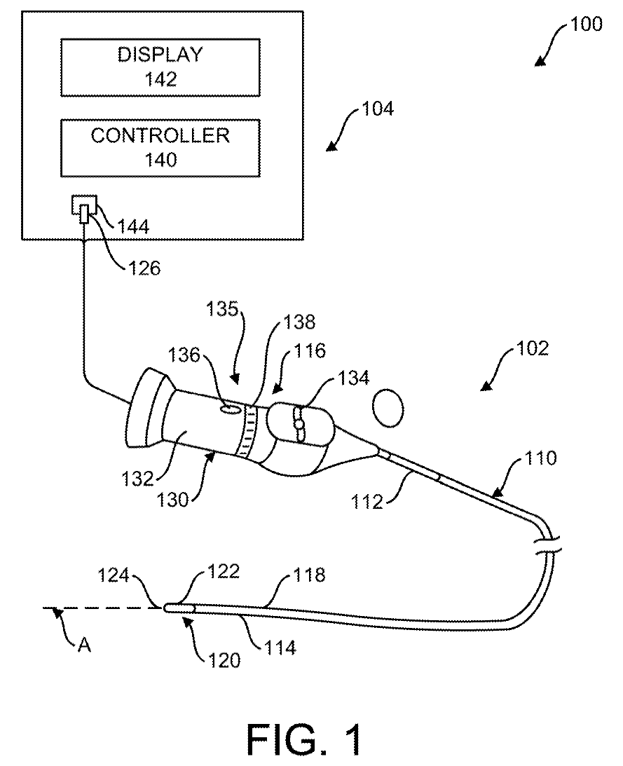

FIG. 1 illustrates an embodiment of a medical imaging system 100 suitable for use in a clinical setting for a medical procedure on a patient, such as a medical procedure, including an electrophysiology procedure or intracardiac procedure, on a heart of the patient. The medical imaging system 100 includes a medical imaging catheter system 102 configured to be inserted into the patient for imaging of portions of the patient’s anatomy and a medical imaging console 104 configured to be coupled to the medical imaging catheter system 102 and generate an image on a device, such as a display. The medical imaging catheter system 100 includes a medical imaging catheter 110, an introducer sheath, and various connecting elements, such as cables and tubing, that connect the components of the medical imaging catheter system 102 to one another and to the components of the medical imaging console 104. The medical imaging system can be employed in transseptal catheterization for several percutaneous interventions, including left heart catheter ablation, atrial septal defect closure for effective alternative to surgical intervention. Access to the patient’s heart can be obtained through a vessel, such as a peripheral vein often in the groin, such as the femoral vein, or possibly in the shoulder or neck. Once access to the vessel is obtained, the medical imaging catheter 110 can be navigated to within the patient’s heart, such as within a chamber of the heart.

The medical imaging catheter 110 includes a proximal end region 112 and a distal end region 114. The medical imaging catheter 110 defines a longitudinal axis A that passes through a centroid of a cross section of the distal end region 114. The proximal end region 112 of the medical imaging catheter 110 includes a catheter hub 116 configured for a clinician to hold and manipulate, and the distal end region 114 is configured and arranged to be inserted into the patient. The medical imaging catheter 110 includes a shaft 118 that is dimensioned to be inserted within regions of the patient that are difficult to navigate, including blood vessels, heart chambers, and, in some cases, the gastrointestinal tract, the urinary tract, and the pulmonary system.

In embodiments, the medical imaging catheter 110 is configured as an ICE device, or ICE catheter. The distal end region 114 includes an imaging module 120 that can be, for example, extended into the patient’s heart and disposed at the end of the shaft 118. In the illustrated embodiment, the imaging module 120 can include an imaging module cover 122 forming a distalmost tip 124, the imaging module cover 122 being mechanically coupled to a distal end region 114 of the shaft 118. The imaging module includes an imaging transducer configured for imaging the patient’s anatomy such as target anatomy proximate the distal end region 114. In embodiments, the imaging module 120 includes an ultrasound imaging device configured to transmit and receive acoustic energy to generate ultrasound images of the target anatomy. The imaging module 120, such as the imaging transducer, is coupled to elongated lead conductors that extend from the distal end region 114 along the catheter shaft 118 to the proximal end region 112, such as to a plug 126 at the proximal end region 112. In one embodiment, the lead conductors include electrically conductive elements, configured to carry an electrical signal, such as wires or traces that are electrically insulated from one another within an insulative sheath, such as with an insulative polymer sheath, or disposed on a flex circuit extending the length of the shaft 118. The plug 126 is configured to be mechanically and electrically coupled to the medical imaging console 104, for example, either directly or via intermediary cabling.

In the illustrated embodiment, the hub 116 includes a housing 130 forming handle assembly 132 and a steering control unit with a steering actuator 134. The steering control unit is provided for articulating the distal end region 114 and positioning the imaging module 120. The steering control unit includes, in some embodiments, steering cables coupled to the steering actuator 134 and disposed within the shaft 118 extending to the distal end region 114. The steering actuator 134 can be rotated to facilitate positioning of the imaging module 120. When the clinician rotates the steering actuator 134, a steering cable pulls the distal end region to bend the shaft 118 to select a position of the imaging module with respect to the target anatomy. The handle assembly 134 also includes mode controls 135 having a mode actuator 136, such as a pushbutton in one embodiment, and an adjuster dial 138, configured for the user to manipulate, and are applied in cooperation with the medical imaging console 104.

The medical imaging console 104 includes a controller 140, such one or more controllers, processors, or computers, that executes instructions or code, such as processor-executable instructions, stored on a non-transitory computer readable medium, such as a memory device, or memory, to cause, such as control or perform, the aspects of the medical imaging catheter system 100. The medical imaging console 104, includes a receptacle 144 configured to couple to cabling or plug 126 and a display 142, or is configured to be coupled to a display 142 via a display connection. In the illustrated embodiment, the controller 140 is configured to receive and respond to a signal from the handle assembly 134 including a signal received from the mode actuator 136 and the adjuster dial 138. The controller 140 is configured to provide a signal, such as a plurality of concurrent or space-apart-time electrical signals, to the connected medical imaging catheter 110 via the receptacle 142 that is transmitted along the lead conductors to the imaging module 120. The controller 140 is also configured to receive a signal, such as a plurality of concurrent or space-apart-time electrical or optical signals, from the connected medical imaging catheter 110 along lead conductors from the imaging module 120. The imaging module 120 includes an ultrasound imaging device configured to transmit and receive acoustic energy to generate ultrasound images with the medical imaging console 104.

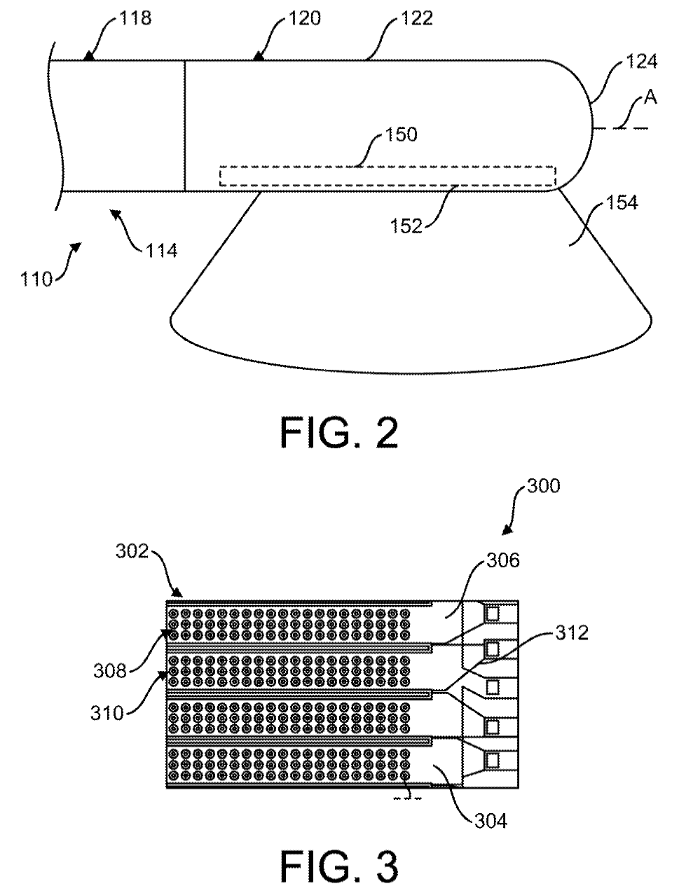

FIG. 2 illustrates an embodiment of the distal end region 114 of the medical imaging catheter 110, which includes the imaging module 120 that can be extended into the patient’s heart and disposed at the end of a shaft 118. In the illustrated embodiment, the imaging module 120 include the imaging module cover 122 forming the distalmost tip 124, although other embodiments are contemplated. The imaging module 120 also includes an ultrasound imaging device 150. The cover 122 encloses the imaging device 150. The cover 122 can be constructed from a material having electrically insulative and low-loss acoustic properties to form an acoustic window, such as an epoxy encapsulant or a polyether block amide (PEBA) such as available under the trade designation PEBAX or a thermoplastic elastomer (TPE) available under the trade name VISTAMID. In one embodiment, the ultrasound imaging device 150 (illustrated in phantom) is disposed within the cover 122 with respect to the longitudinal axis A in such a manner to produce a side image, such as an image perpendicular to the longitudinal axis A. In the illustrated embodiment, the ultrasound imaging device 150 includes an operable or active surface 152 (illustrated in phantom) configured for producing an acoustic signal and receiving echoes that is aligned parallel with the longitudinal axis A and configured to image in a direction perpendicular to the longitudinal axis A.

The ultrasound imaging device 150, in some embodiments, provides a wedge-shaped image, such as a ninety-degree sector plane, from the distal end of the medical imaging catheter 110, such as the side of the distal end region 114 in some embodiments, that can be displayed on the medical imaging console 104. In one example of the ultrasound imaging device 150, a two-dimensional imaging phased array device, which include one-dimensional arrays, creates two-dimensional images by steering an acoustic beam across a two-dimensional plane. Each acoustic beam, or scan line, produces echoes that are measured and combined with other scan lines into an ultrasound image. To create a three-dimensional or four-dimensional (including time as a dimension) image, a two-dimensional array is applied to steer the beam throughout a three-dimensional volume or two orthogonally disposed one-dimensional arrays scan each scan beams across a respective two-dimensional plane. The ultrasound imaging device 150 still produces a wedge-shaped image, such as a ninety-degree sector plane, which is plus/minus forty-five degrees in the axis perpendicular to the longitudinal axis A, as indicated with sector plane 154.

FIG. 3 illustrates an example ultrasound transducer assembly 300 that is adapted for use with the ultrasound imaging device 150 and an ultrasound transducer of the front viewing imaging device 150. The illustrated ultrasound transducer assembly 300 includes a micro-electromechanical system (MEMS) transducer array 302 such as a piezoelectric micro-machined ultrasound transducer (pMUT) or other types of MEMS transducers such as capacitive micromachined ultrasound transducers (“cMUT”), interconnected with a flexible circuit 304 for transmission and electrical interconnects. Other examples of ultrasound transducer assemblies are contemplated. In the illustrated embodiment, the flexible circuit 304 includes a flexible circuit substrate 306 with the transducer array 302, including a plurality of transducer array elements 308 comprising a plurality of transducer cells 310, and associated electrical pathways and connections 312 disposed on the flexible circuit. In the illustrated embodiment, the transducer cells 310 are pMUT cells and are configured to be arranged on an operable or active surface 152. The lead conductors are connected to the ultrasound transducer assembly 300 such as via the electrical pathways and connections 312. Other examples of the ultrasound transducer assembly are contemplated, such as a transducer array and electrical pathways and connections disposed on printed circuit board. Each of the plurality of transducer array elements 308, via the lead conductors, are configured to transmit and receive, ultrasound waves. The ultrasound waves may have a bandwidth including a predetermined fundamental mode vibration of each of the plurality of transducer array elements 308, such that a single array element can transmit and receive multiple fundamental mode vibrations simultaneously. The plurality of transducer array elements 308 transmit and receive the ultrasound waves with respect to the heart or at least a portion of the heart. The transducer cells 310 are arranged in a manner to provide a wide bandwidth of the individual focused wave. In one embodiment, the ultrasound transducer assembly 300 is constructed from a pMUT array containing individual cells 310 of different diameters. In one embodiment, to achieve wider bandwidth with pMUT array elements, multiple diameters of pMUT cells are integrated into one array element. A broader bandwidth is realized through the complex interaction between the individual pMUT elements by arranging pre-shaped pMUTs with different diameters.

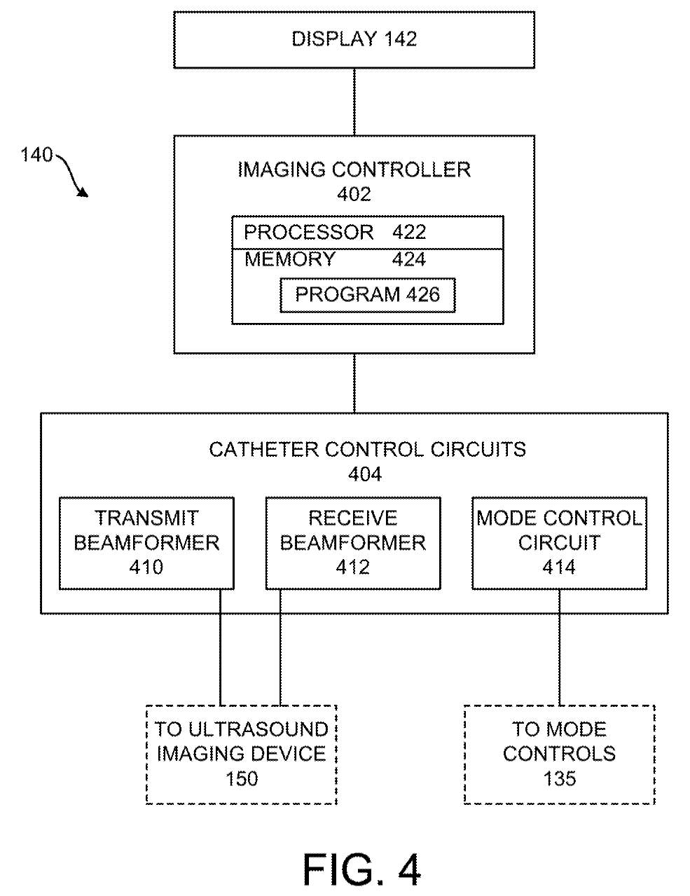

FIG. 4 illustrates an embodiment of the controller 140 of the medical imaging console 104. The controller 140 includes an imaging controller 402 coupled to a catheter control circuit 404. The catheter control circuit 404 is configured to be coupled to and operate with the ultrasound imaging device 150 within the medical imaging catheter 110 such as via exchanging electrical or optical signals and with mode controls 135 on the hub 116. In the illustrated embodiment, the catheter control circuit 404 includes a transmit beamformer 410, a receive beamformer 412, and mode control circuit 414 having mode selector and adjuster circuitry configured to operate with the medical imaging catheter 110.

The imaging controller 402 is implemented with any combination of hardware and programming to receive inputs from and provide outputs to catheter control circuit 404, and to provide outputs to and, in some embodiments, receive inputs from the display 142. In one embodiment, the imaging controller 402 incudes a processor 422 operably coupled to a memory device 424 (a tangible storage medium). The memory device 424 can store processor-executable instructions configured to control the processor 422, such as a program 426. Examples of a memory device 424 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. Memory device 424 can include various combinations of one or both of non-volatile memory devices and volatile memory devices. The processor 422 includes an output port that allows the processor 422 to control or receive inputs from the catheter control circuit 404 and generate a visualization for facilitation on the display 142 according to a programmed scheme.

In other embodiments, the functionalities of imaging controller 402 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, of this disclosure illustrated with the processor 422, memory 424, and program 426 can be implemented in such electronic circuitry.

The catheter control circuit 404 is interposed between the imaging controller 402 and the catheter 110. The transmit beamformer 410 is configured for transmission of an electrical signal or electrical impulse towards the ultrasound imaging device 150 of the medical imaging catheter 110. The receive beamformer 412 is configured to receive an electrical signal or electrical impulse from the ultrasound imaging device 150. The mode control circuit 414 receives electrical signals from the mode controls 135 on the hub 116 and provides associated control signals to the imaging controller 402.

The imaging controller 402 is configured to generate a visualization for facilitation on the display 142. In embodiments, the visualization is based on a mode of an image obtained via the ultrasound imaging device 150. In embodiments, this the visualization is a wedge-shaped image, such as a ninety-degree sector plane, which is plus/minus forty-five degrees in the axis perpendicular to the longitudinal axis A. In one embodiment, the imaging controller 402 is configured to operate in cooperation with the ultrasound imaging device 150 in a plurality of modes, such as an amplitude mode (A-mode), a motion mode (or M-mode), a brightness mode (or B-mode), and a Doppler mode including pulse wave Doppler, continuous wave Doppler, spectral Doppler, and color Doppler. Depending on a medical imaging application, each mode can provide advantages, disadvantages, or particular features that are useful to a clinician. In one example configuration, the visualization is presented in B-mode. In some embodiments of the B-mode configuration, the visualization is a wedge-shaped ultrasound image, such as a ninety-degree sector plane, which is plus/minus forty-five degrees to the longitudinal axis A. In the embodiments, the imaging controller 402 is able to change modes to another mode and present a corresponding visualization. For example, the imaging controller 402 configured to generate a visualization in B-mode can be configured to generate a visualization in pulse wave Doppler mode. In some applications, for instance, certain anatomical features are not well distinguishable in B-mode but are better distinguished in pulse wave Doppler mode. Accordingly, a clinician using B-mode can select pulse wave Doppler mode on the imaging controller, make adjustment to the image, and readily identify the anatomy in the image or maneuver the imaging module 120 to view the anatomy of interest. Once a feature has been distinguished from other features in pulse wave Doppler mode and identified, the clinician can return to a visualization in B-mode for a two-dimensional view of the anatomy.

Mode selection controls and image adjustment controls are typically included on a control panel or user interface of the medical imaging console 104. Operation of the medical imaging console 104, however, such as utilization of all modes and associated adjustments, can be complicated or involve knowledge from special sonography training that many clinicians do not possess. To fully utilize all the features of a medical imaging system 100 during a medical procedure such as an electrophysiology procedure using an ICE catheter 110, the clinician will operate the catheter system 102 while a sonographer will operate the imaging console 104, which requires an additional specialist to attend the procedure. In many circumstances, a clinician will use a small portion of the features of the medical imaging system, and the hub 116 of the present disclosure provides access to the features interest in a readily available and easy to use manner.

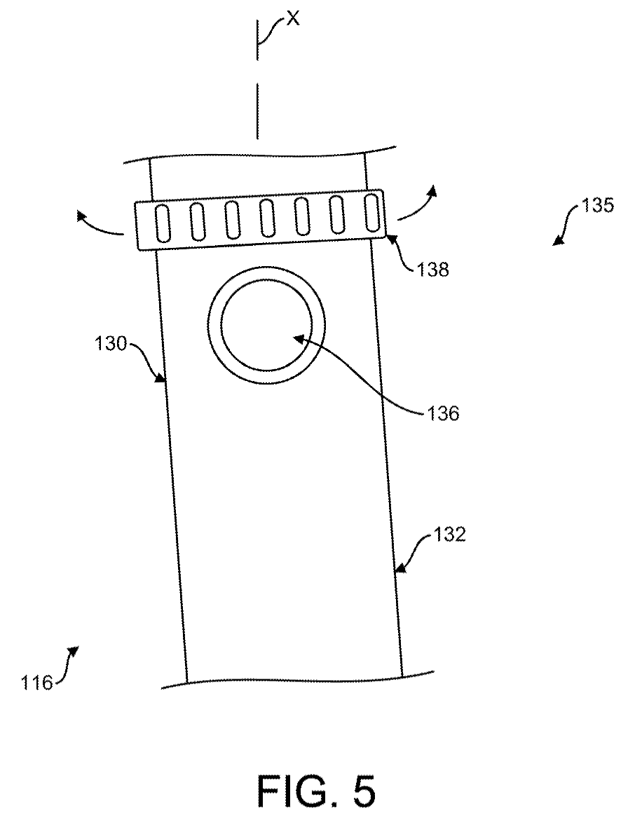

FIG. 5 illustrates the hub 116 of the medical imaging catheter 110 with the handle assembly 132, which is suitable for a clinician to hold in their hand as they maneuver the imaging module and manipulate the mode controls 135. The mode controls 135 include the mode actuator 136 and adjuster dial 138. The mode actuator 136 in the illustrated embodiment includes a pushbutton on the handle assembly 132, and the adjuster dial 138 includes a ring around the handle assembly 132 that is rotatable around an axis X with respect to the handle assembly. In one embodiment, the adjuster dial 138 can be rotated in a first direction about the axis X with respect to the handle assembly 132 to provide first type of adjustment and rotated in a second direction, opposite the first direction, about the axis X to provide a second type of adjustment. The clinician can depress the mode actuator 136 with a thumb or forefinger and rotate the adjuster dial 138 with the thumb and forefinger while holding the handle assembly 132. The mode controls 135 can include electronic elements and circuits within the hub 116 coupled to the mode actuator 136 and adjuster dials, such as a switch and potentiometer, to affect the functions and provide electrical signals to the catheter control circuit 404. In one embodiment, the mode controls include an electronic element within the handle assembly 132 and an actuation mechanism, such as a combination of mode actuator 136 and adjuster dial 138, disposed on the handle assembly 132. Mode controls 135 in the illustrated embodiment include mode actuator 136 and adjuster dial 138 configured as separate actuation mechanisms. In other embodiments, the mode actuator and adjuster dial of mode controls 135 are integrated into the same actuation mechanism. In one particular embodiment, the adjuster dial is a pressure sensitive circuit that responds to a specific input to initiate the mode actuation, such as applying a level of pressure at multiple regions. Other configurations are possible.

The mode controls 135 are configured to allow a user to operate features of the controller 140. The mode controls 135 configured to switch the medical imaging catheter into one of a plurality of modes and to adjust an aspect of the mode. In one embodiment, the controller 140, such as the imaging controller 402, includes a set of operatable features, and the mode controls 135 are configured to operate a subset of the operatable features. In one embodiment, the subset of the operatable features is less features than the full set of operatable features. In one embodiment, an operatable feature of the controller 140, such as the imaging controller 402, includes a feature selectable or adjustable by a user via an interface of the controller 140. In one embodiment, a clinician holds the handle assembly 132 during operation of the catheter 110 and selects features of the imaging controller 402 such as modes via manipulating the mode actuator 136 and then adjusts an adjustable aspect of the selected mode via rotating the adjustment dial 138 with respect to the handle assembly.

In one example, the imaging controller 402, via program 426, is configured to respond to the mode controls. The imaging controller 402 is configured to respond to the mode actuator 136 while the imaging controller is operational and in an initial current mode. A signal from the mode controller 136, such as when mode controller 136 is depressed, causes the imaging controller to switch from the initial mode, or initial feature, to a feature of the subset of the features, such as a first feature. In one embodiment, selection via the mode controller 136, such as depression of the button, toggles the imaging current between the current feature of the imaging controller and the feature in the subset of features. For instance, the subset of features includes one feature, and depression of the button while the controller is in one feature toggles the controller to change into the other feature. In one embodiment, selection via the mode controller 136, such as depression of the button, cycles through the subset of features by switching the imaging controller 402 to the next or subsequent feature in the subset of features, the subset of features arranged in a sequence for selection. For example, from the initial feature, depression of the button causes the controller 140 to change to a first feature of the subset of features; and from the first feature, depression of the button causes the controller 140 to change to a second feature of the subset of features. Of the subset of features includes two features, from the second feature, depression of the button causes the controller 140 to change to the initial feature. Also, the controller 140 responds to an amount of rotation of the adjustment dial to adjust the selected feature. In one embodiment, the subset of features is a subset of modes.

In one embodiment, the initial mode or first feature of a controller 140 is a default mode, such as B-mode. For example, a clinician has selected B-mode of the available modes on the controller 140 for use with the medical imaging catheter in a medical procedure. In this embodiment, a first features is a pulse wave Doppler mode. While in the current mode, i.e., B-mode, the adjuster dial 138 can be rotated to adjust an aspect of the B-mode, such as center frequency. The controller 140 responds to rotation of the adjuster dial 138 in the first direction by increasing the center frequency and responds to rotation of the adjuster dial 138 in the second direction by decreasing the center frequency. While configured in B-mode, the controller 140 responds to the depression of the mode actuator 136 by switching to pulse wave Doppler mode. While in the pulse wave Doppler mode, the adjuster dial 138 can be rotated to adjust an aspect of the selected mode, such as Doppler window. The controller 140 responds to rotation of the adjuster dial 138 in the first direction by increasing the Doppler window and responds to rotation of the adjuster dial 138 in the second direction by decreasing the Doppler window. While configured in the pulse wave Doppler mode, the controller responds to the depression of the mode actuator 136 by switching to B-mode.

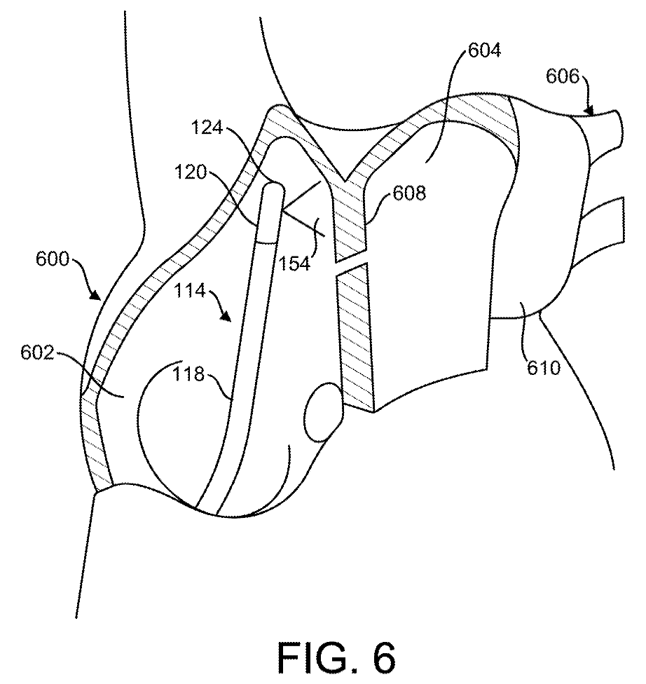

FIG. 6 illustrates the medical imaging catheter 110 after the distal region 114 has been directed through the vasculature and into the right atrium 602 of the patient’s heart 600. In the embodiment, the catheter images regions of the patient’s heart using sector plane 154. Clinicians often image anatomical features close to the left atrium 604, such as pulmonary veins 606, from the catheter 110 in the right atrium 602 and across the intra-atrial septum 608 tissue boundary. Depth of penetration of a 10 MHz ultrasound, however, creates inherent limitations for imaging relatively distant structures, such as the pulmonary veins from the right atrium 602. Accordingly, anatomical features close to the left atrium can be difficult to visualize. While operating the medical imaging system 100 in a B-mode, some clinicians find it difficult to distinguish the left atrial appendage 610 from the pulmonary veins 606. Additionally, some clinicians find it difficult to distinguish the one of the pulmonary veins from each other.

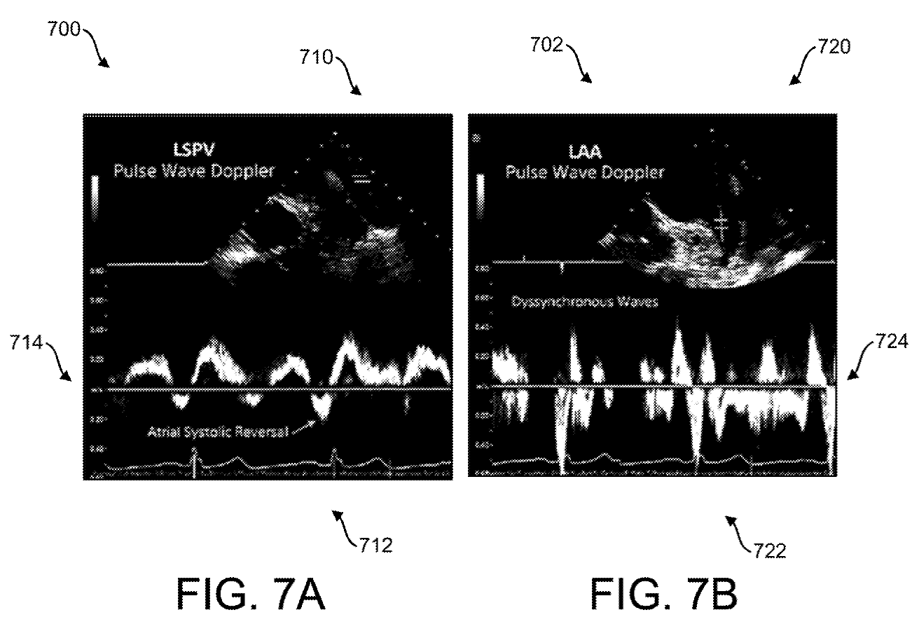

FIGS. 7A, 7B illustrate embodiments of a visualizations 700, 702 facilitated by the controller 140 on the display 142 from images taken by a catheter 110 imaging via sector plane 154 as depicted in FIG. 6. When the controller is configured in B-mode in the illustrated embodiment, the left superior pulmonary vein is difficult to distinguish from the left atrial appendage. The left superior pulmonary vein is relatively easy to distinguish from the left atrial appendage in Doppler mode. FIG. 7A illustrates a visualization 700 of the left superior pulmonary vein including a B-mode section 710 of the visualization 700 and a pulse wave Doppler section 712 of the visualization. FIG. 7B illustrates a visualization 702 of the left atrial appendage including a B-mode section 720 of the visualization 702 and a pulse wave Doppler section 722 of the visualization 702. To distinguish the left superior pulmonary vein from the left atrial appendage, the clinician can depress the mode actuator 136, and the controller 140 responds to the depressed mode actuator 136 by switching from the B-mode to a Doppler mode, such as pulse wave Doppler mode. In the illustrated embodiments of FIGS. 7A, 7B, both the B-mode and the pulse wave Doppler modes are included in a visualization as in separate sections, such as sections 710, 712 of visualization 700 or sections 720, 722 of visualization 702. The clinician can rotate the adjustment dial 138, and the controller 140 responds to the rotated adjustment dial 138 via adjusting the Doppler window. As indicated in a comparison of the visualizations 700, 702, the visualization of the left atrial appendage 702 of FIG. 7B includes dyssynchronous waves 724 in the Doppler section 722 that do not appear in the Doppler section 712 of the left superior pulmonary vein visualization 700 of FIG. 7A. Once the pulmonary vein is identified, the clinician can depress the mode actuator 136, and the controller 140 responds to the depressed mode actuator 136 by switching from the pulse wave Doppler mode back to B-mode. The clinician can rotate the adjustment dial 138, and the controller 140 responds to the rotated adjustment dial 138 via adjusting the center frequency. Thus, using the mode controller 135 on the hub 116, the clinician can identify an anatomical feature of the heart and quicky change from a B-mode to a Doppler mode and return to the B-mode once a feature is identified without the aid of a sonographer. Further, the Doppler section 712 of the left superior pulmonary vein visualization 700 indicates an atrial systolic reversal 714, which can be indicative of congestive heart failure, which can quickly be identified via using the mode controls 135 to include a Doppler section 712 with the B-mode section 710 and is not readily identifiable via B-mode.

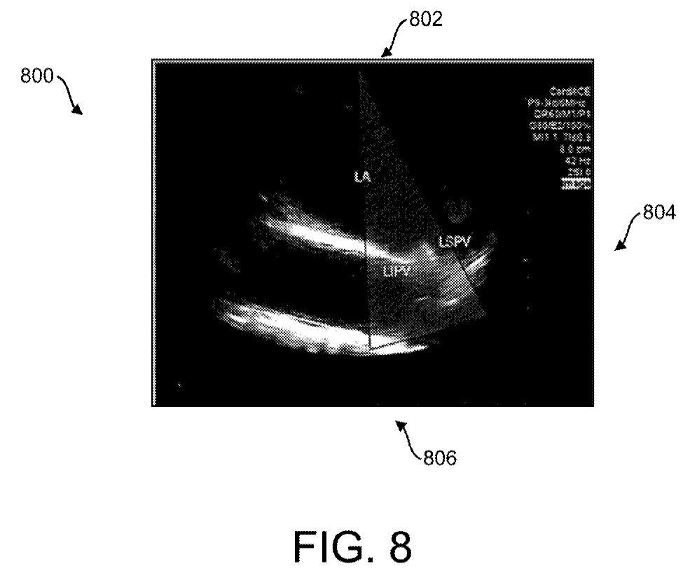

In one embodiment, a feature in the subset of features can include an anatomical feature detector mode, such as a pulmonary vein detector mode. FIG. 8 illustrates an embodiment of a visualization 800 facilitated by the controller 140 on the display 142 from an image of the pulmonary veins 606 taken by the catheter 110 imaging via sector plane 154 as depicted in FIG. 6. The image of the pulmonary veins 606 in visualization 800 is presented in B-mode. the clinician can depress the mode actuator 136 or repeatedly depress the mode actuator 136, and the controller 140 responds the depressed mode actuator 136 via switching to or cycling through modes to the pulmonary vein detector mode. Once in the pulmonary vein detector mode, the ultrasound image is augmented to improve image quality or features within the image are identified, each via applying an artificial intelligence program such as machine learning using image libraries to the ultrasound image. In one embodiment, the ultrasound image is included in the imaging library. Using the artificial intelligence program, features of the ultrasound image can be automatically identified and displayed on the visualization to aid the clinician in determining anatomical features, such as distinguishing pulmonary veins, via labels 802, 804, 806 such as labels indicating the left atrium 802, the left superior pulmonary vein 804, and the left inferior pulmonary vein 806. After the anatomical features are identified, the clinician can depress the mode actuator 136, and the controller 140 responds to the depressed mode actuator 136 by switching from the anatomical feature detector mode back to B-mode.

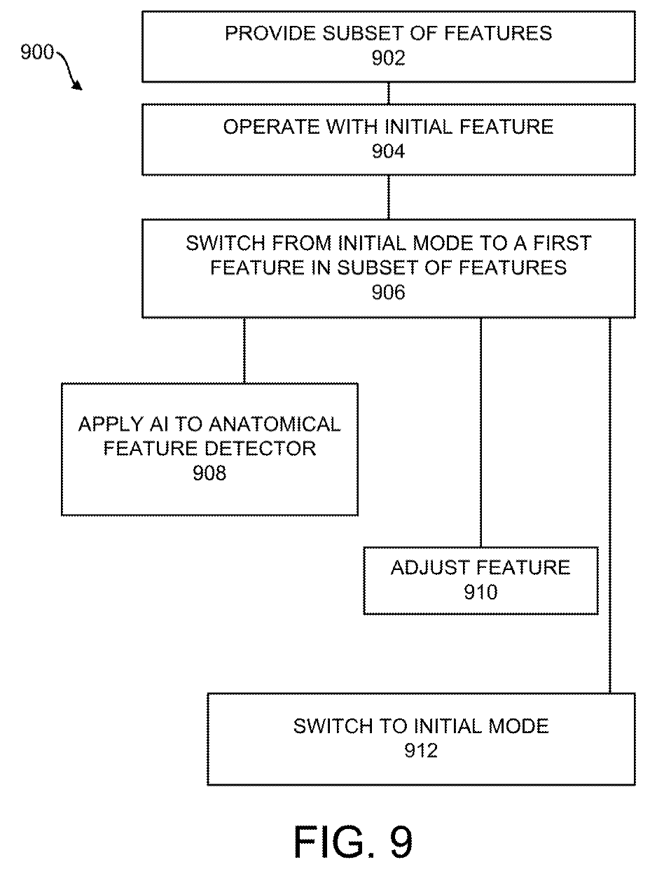

FIG. 9 illustrates an example method 900, which can be implemented with the controller 140 of the medical imaging console 104, such as via a set of executable instructions in program 426 of FIG. 4, in which the medical imaging console 104 is coupled to the medical imaging catheter 110. The method 900 provides a designated subset of features 902 from the full set of features of the controller 140. The subset of features includes one or a plurality of features. The method 900 associates an adjustment with each feature of the subset of features. In some embodiments, the subset of features includes less than the full set of features. In some embodiments, the subset of features includes user-selected features, or features that a user designates to be included in the subset of features. In some embodiments, the program 426 provides a plurality of subsets of features, such a different combination of features for a plurality of medical procedures. A user can select the medical procedure, and the program 426 applies a subset of features from the plurality of subsets of features associated with the medical procedure. The controller 140 operates with an initial feature at 904, such as the controller 140 generates a visualization facilitated by the controller 140 on the display 142 from an image by the catheter 110 applying the initial feature to the image. The method 900 responds to a mode actuator signal, such as a signal provided from a depressed mode actuator 136, and switches from the initial mode to the feature of the subset of features, switches from the initial mode to a first features in a subset of a plurality of features, or switches from the first features to a second features in a subset of a plurality of features at 906. If the switched-to feature is an anatomical feature detector at 908, an artificial intelligence program is applied to the anatomical feature detector 908. The method 900 responds to an adjustment signal, such as signal provided from an adjustment dial 138, and makes an associated adjustment based on an amount of signal at 910. The method 900 responds to a mode actuator signal, such as a signal provided from a depressed mode actuator 136, and switches to the initial mode at 912.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical imaging catheter configured to be extended into a patient’s heart, the medical imaging catheter comprising:
a shaft having a proximal region and an opposite distal region;
an ultrasound imaging module coupled to the distal region and configured to be extended into the patient’s heart; and
a handle assembly coupled to the proximal region, the handle assembly including a mode controller configured to switch the medical imaging catheter into one of a plurality of modes and to adjust an aspect of the mode.

2. The medical imaging catheter of claim 1, wherein the medical imaging catheter is an intracardiac echocardiography (ICE) device.

3. The medical imaging catheter of claim 1, wherein the mode controller includes a mode actuator and an adjuster dial.

4. The medical imaging catheter of claim 3, wherein the mode actuator includes a pushbutton and the adjuster dial includes a ring rotatable with respect to the handle assembly.

5. The medical imaging catheter of claim 1, wherein the medical imaging catheter is configured to be coupled to a medical imaging console as a medical imaging system.

6. The medical imaging catheter of claim 5, wherein the medical imaging console includes controller configured to apply a plurality of features to an ultrasound image.

7. The medical imaging catheter of claim 6, wherein the controller is configured to respond to a signal from the mode controller.

8. The medical imaging catheter of claim 7, wherein the mode controller switches between a subset of the plurality of features.

9. The medical imaging catheter of claim 8, wherein the mode controller toggles between an initial feature and another feature.

10. The medical imaging catheter of claim 9, wherein the initial feature is a brightness mode and the another feature is a Doppler mode.

11. The medical imaging catheter of claim 10, wherein the mode controller is configured to adjust a center frequency while the controller is configured in the brightness mode and configured to adjust a Doppler window while the controller is configured in the Doppler mode.

12. The medical imaging catheter of claim 9, wherein the subset of features is less than the plurality of features.

13. The medical imaging catheter of claim 6, wherein the plurality of features includes a plurality of ultrasound modes.

14. The medical imaging catheter of claim 6, wherein the plurality of features includes an anatomical feature detector mode configured to automatically determine an anatomical feature from an ultrasound image via an artificial intelligence.

15. The medical imaging catheter of claim 14, wherein the anatomical feature detector mode includes a pulmonary vein detector mode configure to apply labels to a visualization of the pulmonary veins from an ultrasound image.

16. An intracardiac echocardiography (ICE) catheter configured to be extended into a patient’s heart, the ICE catheter comprising:
a shaft having a proximal region and an opposite distal region;
an ultrasound imaging module coupled to the distal region and configured to be extended into the patient’s heart, the ultrasound imaging module including a piezoelectric micro-machined ultrasound transducer (pMUT) connected to a flex circuit configured for transmission of electrical signals along the shaft; and
a handle assembly coupled to the proximal region, the handle assembly including a mode controller configured to switch the ICE catheter into one of a plurality of modes and to adjust an aspect of the mode, the mode controls including an electronic element within the handle assembly and an actuation mechanism disposed on the handle assembly.

17. The ICE catheter of claim 16, wherein the mode controller includes a mode actuator and an adjuster dial.

18. The ICE catheter of claim 17, wherein the mode actuator includes a pushbutton and the adjuster dial includes a ring rotatable with respect to the handle assembly.

19. A method for use with a medical imaging system having a medical imaging catheter having an ultrasound imaging module configured to be extended into a patient’s heart and a handle assembly coupled to the imaging module, the handle assembly including a mode controller having a mode actuator and an adjuster dial, the method comprising:
applying a plurality of features to an ultrasound image, the plurality of features including a brightness mode and a Doppler mode; and
switching between a subset of the plurality of features in response to a signal from the mode actuator; and
adjusting a center frequency while in the brightness mode and adjusting a Doppler window while in the Doppler mode in response to a signal from the adjuster dial.

20. The method of claim 19, wherein the plurality of features includes an anatomical feature detector mode configured to automatically determine an anatomical feature from an ultrasound image via an artificial intelligence.
